SEQUENCE LISTING (8 sequences)

SEQ ID NO: 1 - DNA, 7 bp, Artificial Sequence
  Synthetic bar code sequence
nnnnnnn

SEQ ID NO: 2 - DNA, 20 bp, Artificial Sequence
  Primer sequence
ctgccctcaacaagatgttt

SEQ ID NO: 3 - DNA, 20 bp, Artificial Sequence
  Primer sequence
ctgccctcaacaagatgttt

SEQ ID NO: 4 - DNA, 19 bp, Artificial Sequence
  Primer sequence
aatcaacccacagctgcac

SEQ ID NO: 5 - DNA, 18 bp, Artificial Sequence
  Primer sequence
agatttggacctgcgagc

SEQ ID NO: 6 - DNA, 20 bp, Artificial Sequence
  Primer sequence
gagcggctgtctccacaagt

SEQ ID NO: 7 - DNA, 20 bp, Artificial Sequence
  Primer sequence
aatgatacggcgaccaccga

SEQ ID NO: 8 - DNA, 21 bp, Artificial Sequence
  Primer sequence
caagcagaagacggcatacga

(19) United States
(12) Patent Application Publication
(10) Pub. No.: US 2015/0126376 A1
(43) Pub. Date: May 7, 2015

(54) COMPOSITIONS AND METHODS FOR SENSITIVE MUTATION DETECTION IN NUCLEIC ACID MOLECULES

(71) Applicant: Fred Hutchinson Cancer Research Center, Seattle, WA (US)

(72) Inventors: Jason H. Bielas, Seattle, WA (US); Nolan G. Ericson, Seattle, WA (US)

(21) Appl. No.: 14/407,439

(22) PCT Filed: Jun. 14, 2013
(86) PCT No.: PCT/US2013/046011

§ 371 Date: Dec. 11, 2014

Related U.S. Application Data
(60) Provisional application No. 61/659,837, filed on Jun. 14, 2012.

(51) Int. Cl. C12Q 1/68

(52) U.S. Cl. CPC C12Q 1/6874
USPC 506/2

(57) ABSTRACT

The present disclosure provides methods for detecting mutations in a target nucleic acid molecule by rolling circle amplification of a library of double-stranded circular bar-coded template molecules. Also provided herein are methods for enriching a target nucleic acid molecule.

### CROSS REFERENCE TO RELATED APPLICATIONS

This application claims the benefit under 35 U.S.C. §119(e) to U.S. Provisional Application No. 61/659,837 filed on Jun. 14, 2012, which application is incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 360056_{—}414WO_SEQUENCE_LISTING.TXT. The text file is 2.1 KB, was created on Jun. 12, 2013 and is being submitted electronically via EFS-Web.

### BACKGROUND

1. Technical Field

The present disclosure relates to compositions and methods for accurately detecting mutations in a target nucleic acid molecule using rolling circle amplification on uniquely tagged double stranded nucleic acid molecules.

2. Description of the Related Art

Circulating cell free DNA extracted from plasma or other body fluids may be exploited as biomarkers for early detection of cancer, assessing prognosis, and monitoring efficacy of anticancer treatment (Gormally et al., 2007, Mutat. Res. 635:105-117; Diehl et al., Proc. Natl. Acad. Sci. USA 2005, 102:16368-16373; Diehl et al., 2008, Nat. Med. 985-990; Schwarzenbach et al., 2011, Nat. Rev. Cancer 11:426-437; Swisher et al., 2005, Am. J. Obstet. Gynecol. 193:662-667; Board et al., 2010, Breast Cancer Res. Treat., 2010, 120:461-467; Yung et al., 2009, Clin. Cancer Res. 15:2076-2084). Characterization of tumor mutation profiles may be beneficial for predicting patient response to therapy, given that biological agents target specific pathways and tumor resistance may be modulated by specific mutations (Banerjee and Kaye, 2011, Eur. J. Cancer 47:S116-S130; Keedy et al., 2011, J. Clin. Oncol. 29:2121-2127; Matulonis et al., 2011, PLoS One 6:e24433; Engelman et al., 2008, Nat. Med. 14:1351-1356). However, genetic heterogeneity is observed between metastatic tumor cells and primary tumor cells and among different metastases (Campbell et al., 2010, Nature 467:1109-1113; Shah et al., 2009, Nature 461:809-813). Evolutionary changes within the cancer can alter the tumor mutational profile and its responsiveness to therapies, which may necessitate serial monitoring of tumor genotypes (Inukai et al., 2006, Cancer Res. 66:7854-7858; Edwards et al., 2008, Nature 451:1111-1115; Maheswaran et al., 2008, N. Engl. J. Med. 359:366-377; Norquist et al., 2011, J. Clin. Oncol. 29:3008-3015). Biopsies are invasive and expensive, and only gives a snapshot of tumor diversity at that particular time and from that particular specimen. For some applications, characterizing individual circulating tumor cells in blood may serve as a “liquid biopsy” that could potentially replace invasive biopsies for assessing molecular changes in tumor cells (Diehl et al., Proc. Natl. Acad. Sci. USA 2005, 102:16368-16373; Diehl et al., 2008, Nat. Med. 985-990; Schwarzenbach et al., 2011, Nat. Rev. Cancer 11:426-437; Swisher et al., 2005, Am. J. Obstet. Gynecol. 193:662-667; Board et al., 2010, Breast Cancer Res. Treat., 2010, 120:461-467; Yung et al., 2009, Clin. Cancer Res. 15:2076-2084). Sensitive methods for detecting cancer mutations in circulating free DNA in plasma or serum may be used for early detection screening (Gormally et al., 2007, Mutat. Res. 635:105-117), prognosis, monitoring tumor dynamics during course of disease, or detection of residual tumors (Diehl et al, 2008, Nat. Med. 14:985-990; Leary et al., 2010, Sci. Transl. Med. 2:20ra14; McBride et al., 2010, Genes Chromosomes Cancer 40:1062-1069). TP53 tumor suppressor gene mutations have been observed in 97% of high grade serous ovarian carcinomas (Ahmed et al., 2010, J. Pathol. 221:49-56; Cancer Genome Atlas Research Network, 2011, Nature 474:609-615). However, TP53 mutations are widespread throughout the whole gene and many mutations are poorly represented or underreported. A non-invasive, cost-effective method for detecting and measuring allele frequency of TP53 genes may be a useful biomarker for high grade serous ovarian carcinomas (Bast, 2011, Ann. Oncol. 22 (Suppl. 8) viii5-viii15; Forshew et al., 2012, Sci. Transl. Med. 4:136ra68).

Circulating DNA is fragmented to an average length of 140 to 170 base pairs, with only several thousand fragments present per milliliter of plasma, and the number of mutant DNA fragments compared to normal circulating DNA is small, sometimes less than 0.1%, making reliable detection challenging (Diehl et al, 2005, Proc. Natl. Acad. Sci. USA 102:16368-16373; Diehl et al., 2008, Nat. Med. 14:985-990; Chan et al., 2008, Clin. Cancer Res. 14:4141-4145; Fan et al., 2010, Clin. Chem. 56:1279-1286; Lo et al., 2010, Sci. Transl. Med. 2:61ra91). Assays have been developed to detect extremely rare alleles in circulating free DNA (Gormally et al., 2007, Mutat. Res. 635:105-117; Diehl et al., Proc. Natl. Acad. Sci. USA 2005, 102:16368-16373; Board et al., 2010 Breast Cancer Res. Treat. 120:461-467; Yung et al., 2009, Clin. Cancer Res. 15:2076-2084; Chen et al., 2009, PLoS One 4:e7220; Kinde et al., 2011, Proc. Natl. Acad. Sci. USA 108:9530-9535; Li et al., 2008, Nat. Med. 14:579-584) and can query predefined or mutational hotspots. However, these assays query individual or few loci rather than the whole gene and have limited ability to detect mutations in genes that lack mutation hotspots, such as TP53 and PTEN tumor suppressor genes (Forbes et al., 2011, Nucleic Acids Res. 39:D945-D950).

### BRIEF SUMMARY

In one aspect, the present disclosure provides a method for detecting mutations in a target nucleic acid molecule, the method comprising: a) a first amplification step comprising rolling circle amplification of a library of double-stranded circular bar-coded template molecules with a first sense primer and a first anti-sense primer specific for a first target nucleic acid molecule, wherein the library of double-stranded circular bar-coded template molecules comprises vectors containing a plurality of double-stranded nucleic acid molecules, wherein each double-stranded nucleic acid molecule is flanked by a 5′ cypher and a 3′ cypher within the vector, wherein the 5′ cypher is different than the 3′ cypher for each double-stranded nucleic acid molecule, and wherein rolling circle amplification produces two complementary strands of tandem nucleic acid molecules comprising multiple copies of first target nucleic acid molecule or portion thereof; b) a second amplification step comprising amplification of the first target nucleic acid molecules or portions thereof and flanking 5′ and 3′ cyphers on each strand of tandem nucleic acid molecules produced from step a); and c) sequencing the first target nucleic acid molecules or portions thereof produced from step b), thereby detecting mutations in the first target nucleic acid molecule compared to a reference first target nucleic acid molecule sequence.

In some embodiments, the plurality of double-stranded nucleic acid molecules is genomic DNA or mitochondrial DNA.

In some embodiments, the first sense primer and the first anti-sense primer specific for the first target nucleic acid molecule each further comprises a tag molecule, wherein the tag molecule may be biotin.

In some embodiments, the method comprises amplifying with a plurality of sense and antisense primers specific for a plurality of different target nucleic acid molecules.

In some embodiments, the target nucleic acid molecule comprises a tumor suppressor gene or an oncogene. In still further aspects, the target nucleic acid molecule comprises BCR-ABL, RAS, RAF, MYC, P53, ER (Estrogen Receptor), HER2, EGFR, mTOR, PI3K, AKT, VEGF, ALK, pTEN, RB, DNMT3A, FLT3, NPM1, IDH1, or IDH2.

In another aspect, the present disclosure provides a method for enriching a target nucleic acid molecule, comprising: a first amplification step comprising rolling circle amplification of a library of double-stranded circular bar-coded template molecules with a first sense or antisense primer specific for a first target nucleic acid molecule, wherein the library of double-stranded circular bar-coded template molecules comprises vectors containing a plurality of double-stranded nucleic acid molecules, wherein each double-stranded nucleic acid molecule is flanked by a 5′ cypher and a 3′ cypher within the vector, wherein the 5′ cypher is different than the 3′ cypher for each double stranded nucleic acid molecule, and wherein rolling circle amplification produces a strand of tandem nucleic acid molecules comprising multiple copies of the first target nucleic acid molecule or portion thereof, thereby enriching the target nucleic acid molecule.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE SEVERAL VIEWS OF THE DRAWINGS

FIG. 1 is a cartoon overview of a portion of an exemplary method of the present disclosure for detecting mutations in a target nucleic acid molecule. Step 1 shows among a plurality of double-stranded nucleic acid molecules, target nucleic acid molecule A and target nucleic acid molecule B, and a plurality of sense and antisense primers specific for target A and target B. Step 2 shows a library of double-stranded circular bar-coded template molecules comprising vectors containing the plurality of double-stranded nucleic acid molecules. Each double-stranded nucleic acid molecule is flanked by a 5′ cypher and a 3′ cypher within the vector, and the 5′ cypher is different from the 3′ cypher for each double-stranded nucleic acid molecule. Specific sense and antisense primers for Target A prime rolling circle amplification of two complementary strands of tandem nucleic acid molecules comprising multiple copies of target A nucleic acid molecule or a portion thereof and the flanking 5′ and 3′ cyphers and vector. Target B specific sense and antisense primers prime rolling circle amplification of two complementary strands of tandem nucleic acid molecules comprising multiple copies of target B nucleic acid molecule or a portion thereof and the flanking 5′ and 3′ cyphers and vector. Step 3 shows a second amplification step comprising amplification of target A nucleic acid molecules or portions thereof and the flanking 5′ and 3′ barcodes from each strand (produced from step 2). Step 3 also shows amplification of target B nucleic acid molecules or portions thereof and the flanking 5′ and 3′ barcodes from each strand. The amplicons produced from step 3 may be sequenced, thereby detecting mutations in target A nucleic acid molecules or target B nucleic acid molecules, when compared to a reference target A sequence or reference target B sequence.

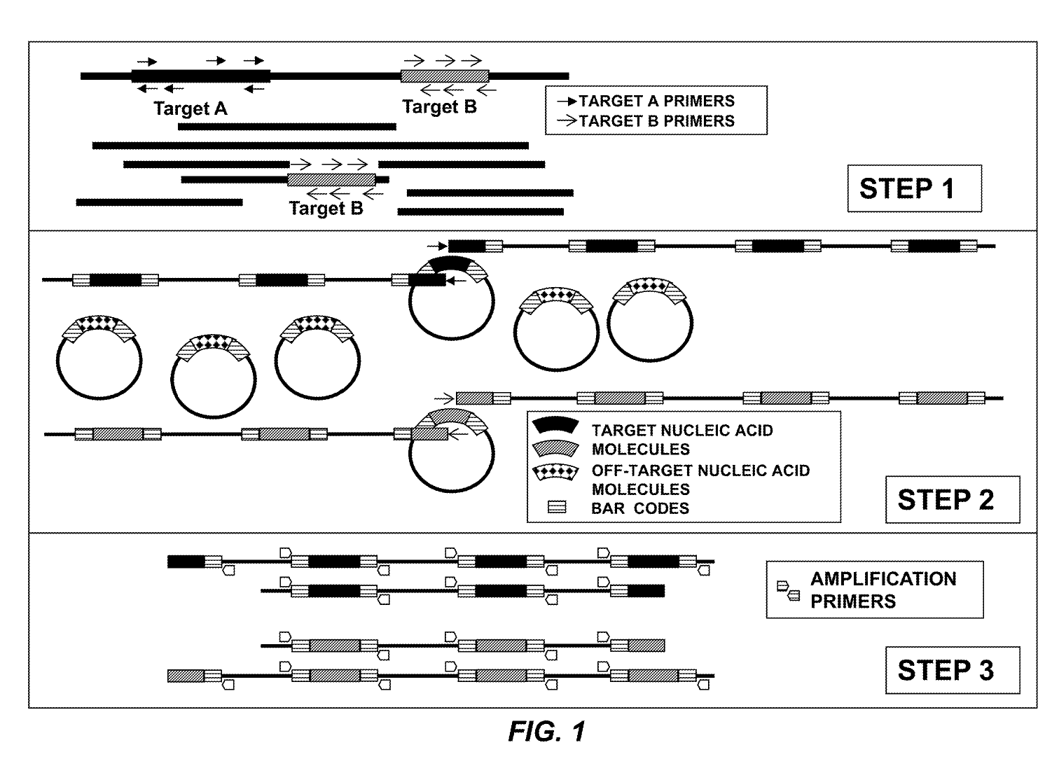

FIG. 2 shows target enrichment of p53 exon 4 containing CyperSEQ vector library molecules by Rolling Circle Amplification (RCA).

### DETAILED DESCRIPTION

In one aspect, the present disclosure provides a method of detecting mutations in a target nucleic acid molecule. A first amplification step comprising rolling circle amplification is performed on a library of double-stranded circular bar-coded template molecules with a first sense primer and a first antisense primer specific for a first target nucleic acid molecule. The library of double-stranded circular bar-coded template molecules comprises vectors containing a plurality of double-stranded nucleic acid molecules, which are each flanked by a 5′ cypher and a 3′ cypher within the vector, and wherein the 5′ cypher is different than the 3′ cypher for each double-stranded nucleic acid molecule. Rolling circle amplification produces two complementary strands of tandem nucleic acid molecules comprising multiple copies of first target nucleic molecule or portion thereof. A second amplification step using the rolling circle amplification products as template amplifies the first nucleic acid molecules or portions thereof, including the flanking 5′ and 3′ cyphers. The amplicons from the second amplification step are sequenced, thereby detecting mutations in the first target nucleic acid molecule compared to a reference first target nucleic acid molecule sequence. By tagging double-stranded nucleic acid molecules with unique cyphers, sequence data obtained from each repeat of target nucleic acid molecule or portion thereof on one strand of rolling circle amplification product can be connected with each other and with the original target nucleic acid molecule. The unique cypher on each strand also allows each repeat of target nucleic acid molecule or portion thereof on one strand of rolling circle amplification product to be linked with each repeat of target nucleic acid molecule or portion thereof on the complementary strand, so that each repeated sequence within a strand and on its complementary strand serves as an internal control. Furthermore, sequence data obtained from one end of a double-stranded target nucleic acid molecule can be specifically linked to sequence data obtained from the opposite end of that same double-stranded target nucleic acid molecule (if, for example, it is not possible to obtain sequence data across the entire target nucleic acid molecule of the library).

The compositions and methods of this disclosure allow a person of skill in the art to more accurately distinguish true mutations (i.e., naturally arising in vivo mutations to a nucleic acid molecule) from artifact “mutations” (i.e., ex vivo mutations to a nucleic acid molecule that may arise for various reasons, such as a downstream amplification error, a sequencing error, or physical or chemical damage). For example, if a mutation pre-existed in the original double-stranded nucleic acid molecule before isolation, amplification or sequencing, then a transition mutation of adenine (A) to guanine (G) identified on one strand will be complemented with a thymine (T) to cysteine (C) transition identified on the other strand. In contrast, artifact “mutations” that arise later in an individual (separate) DNA strand due to polymerase errors during isolation, amplification or sequencing are extremely unlikely to have a matched base change in the complementary strand. The approach of this disclosure provides compositions and methods for interrogating one or more regions within a target nucleic acid molecule, or interrogating one or more target nucleic acid molecules in a multiplex reaction and distinguishing systematic errors (e.g., polymerase read fidelity errors) and biological errors (e.g., chemical or other damage) from actual known or newly identified mutations or single nucleotide polymorphisms (SNPs).

By way of background, any spontaneous or induced mutation will be present in both strands of a native genomic, double-stranded DNA molecule. Hence, such a mutant DNA template amplified using error-free PCR would result in a PCR product in which 100% of the molecules produced by PCR include the mutation. In contrast to an original, spontaneous mutation, a change due to polymerase error will only appear in one strand of the initial template DNA molecule (while the other strand will not have the artifact mutation). If all DNA strands in a PCR reaction are copied equally efficiently, then any polymerase error that emerged at the first PCR cycle likely will be found in at least 25% of the total PCR product. But DNA molecules or strands are not copied equally efficiently, so DNA sequences amplified from the strand that incorporated an erroneous nucleotide base during the initial amplification might constitute more or less than 25% of the population of amplified DNA sequences depending on the efficiency of amplification. Similarly, any polymerase error that occurs in later PCR cycles will generally represent an even smaller proportion of PCR products (i.e., 12.5% for the second cycle, 6.25% for the third, etc.). PCR-induced mutations may be due to polymerase errors or due to the polymerase bypassing damaged nucleotides, thereby resulting in an error (see, e.g., Bielas and Loeb, Nat. Methods 2:285-90, 2005). For example, a common change to DNA is the deamination of cytosine, which is recognized by Taq polymerase as a uracil and results in a cytosine to thymine transition mutation (Zheng et al., Mutat. Res. 599:11-20, 2006)—that is, an alteration in the original DNA sequence may be detected when the damaged DNA is sequenced, but such a change may or may not be recognized as a sequencing reaction error or due to damage arising ex vivo (e.g., during or after nucleic acid isolation).

Due to potential artifacts and alterations of nucleic acid molecules arising from isolation, amplification and sequencing, the accurate identification of true somatic DNA mutations is difficult when sequencing amplified nucleic acid molecules. Consequently, evaluation of whether certain mutations are related to, or are a biomarker for, various disease states (e.g., cancer) or aging becomes confounded.

Next generation sequencing has opened the door to sequencing multiple copies of an amplified single nucleic acid molecule—referred to as deep sequencing. The thought on deep sequencing is that if a particular nucleotide of a nucleic acid molecule is sequenced multiple times, then one can more easily identify rare sequence variants or mutations. In fact, however, the amplification and sequencing process has a fixed error rate, so no matter how few or how many times a nucleic acid molecule is sequenced, a person of skill in the art cannot distinguish a polymerase error artifact from a true mutation.

While being able to sequence many different DNA molecules collectively is advantageous in terms of cost and time, the price for this efficiency and convenience is that various PCR errors complicate mutational analysis.

Disclosed herein is a method for detecting mutations in a target nucleic acid molecule, which utilizes rolling circle amplification on a library of vectors containing a plurality of bar-coded, double stranded nucleic acid molecules, using target nucleic acid molecule-specific primers to selectively amplify the target nucleic acid molecule for sequence analysis. Since rolling circle amplification copies from the same circular template molecule with each round or cycle, it circumvents the clonal amplification of polymerase errors observed in successive PCR cycles. The unique cyphers flanking each copy of the target nucleic acid molecule or portion thereof allows a person of skill in the art to accurately distinguish a polymerase error artifact from a true mutation.

Prior to setting forth this disclosure in more detail, it may be helpful to an understanding thereof to provide definitions of certain terms to be used herein. Additional definitions are set forth throughout this disclosure.

In the present description, the terms “about” and “consisting essentially of” mean±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

A “nucleic acid molecule mutation” or “mutation” refers to a change in the nucleotide sequence of a nucleic acid molecule. A mutation may be caused by radiation, viruses, transposons, mutagenic chemicals, errors that occur during meiosis or DNA replication, or hypermutation. A mutation can result in several different types of change in sequence, including substitution, insertion or deletion of nucleotide(s).

A “nucleic acid molecule” refers to a single- or double-stranded linear or circular polynucleotide containing either deoxyribonucleotides or ribonucleotides that are linked by 3′-5′-phosphodiester bonds. A nucleic acid molecule includes a genomic DNA molecule or a mitochondrial DNA molecule.

As used herein, “target nucleic acid molecule” and variants thereof refer to a nucleic acid molecule or fragments thereof that are subject of a query of mutational status or mutational spectrum. Target nucleic acid molecule includes genes or fragments thereof (e.g., domains, exons, introns, UTRs), coding or non-coding sequence. Target nucleic acid fragments may be generated from longer molecules using a variety of techniques known in the art, such as by mechanical shearing or by specific cleavage with restriction endonucleases.

As used herein, a “library of double-stranded circular bar-coded template molecules” refers to a collection of double-stranded nucleic acid molecule sequences or fragments, including target nucleic acid molecules, that are incorporated into a vector, which may be transformed or transfected into an appropriate host cell. The target nucleic acid molecules of this disclosure may be introduced into a variety of different vector backbones (such as plasmids, cosmids, viral vectors, or the like) so that recombinant production of a nucleic acid molecule library can be maintained in a host cell of choice (such as bacteria, yeast, mammalian cells, or the like). The double-stranded nucleic acid molecules that are incorporated into a vector may be from natural samples (e.g., a genome), or the nucleic acid molecules may be synthetic samples, recombinant samples, or a combination thereof. Prior to insertion into the vector, a plurality of nucleic acid molecules may undergo additional reactions for optimal cloning, such as mechanical shearing or specific cleavage with restriction endonucleases.

For example, a collection of nucleic acid molecules representing the entire genome is called a genomic library. Methods for construction of nucleic acid molecule libraries are well known in the art (see, e.g., Current Protocols in Molecular Biology, Ausubel et al., Eds., Greene Publishing and Wiley-Interscience, New York, 1995; Sambrook et al., Molecular Cloning: A Laboratory Manual, 2nd Ed., Cold Spring Harbor Laboratory Vols. 1-3, 1989; Methods in Enzymology, Vol. 152, Guide to Molecular Cloning Techniques, Berger and Kimmel, Eds., San Diego: Academic Press, Inc., 1987).

Depending on the type of library to be generated, the ends of the double-stranded nucleic acid molecules may have overhangs or be “polished” (i.e., blunted). Together, the double-stranded nucleic acid molecules can be, for example, cloned directly into a vector to generate a vector library, or be ligated with adapters (e.g., adapters comprising unique 5′ and 3′ cyphers). In certain embodiments, double-stranded nucleic acid molecules are cloned into vectors, with a unique 5′ cypher and a unique 3′ cypher or a unique 5′-3′ cypher pair flanking the cloning site. The double-stranded nucleic acid molecules, which are the nucleic acid molecules of interest for amplification and sequencing, may range in size from a few nucleotides (e.g., 15) to many thousands (e.g., 10,000). Preferably, the double-stranded nucleic acid molecules in the library range in size from about 100 nucleotides to about 3,000 nucleotides or from about 150 nucleotides to about 2000 nucleotides.

As used herein, a “nucleic acid molecule primer” or “primer” and variants thereof refers to short nucleic acid sequences that a DNA polymerase can use to begin synthesizing a complementary DNA strand of the molecule bound by the primer. A primer sequence can vary in length from 5 nucleotides to about 50 nucleotides in length, from about 10 nucleotides to about 35 nucleotides, and preferably are about 15, 16, 17, 18, 19, 20, 21, 22, 23, 24, 25, 26, 27, 28, 29, 30, 31, 32, 33, 34, or 35 nucleotides in length. In certain embodiments, a nucleic acid molecule primer that is complementary to a target nucleic acid of interest can be used to initiate an amplification reaction, a sequencing reaction, or both.

As used herein, the term “random cypher” or “cypher” or “bar code” or “identifier tag” and variants thereof are used interchangeably and refer to a nucleic acid sequence comprised of about 5 to about 50 nucleotides in length. In certain embodiments, all of the nucleotides of the cypher are not identical (i.e., comprise at least two different nucleotides) and optionally do not contain three contiguous nucleotides that are identical. In further embodiments, the cypher is comprised of about 5 to about 15 nucleotides, preferably about 6 to about 10 nucleotides, and even more preferably 6, 7, or 8 nucleotides. The library of double-stranded circular template molecules includes 5′ and 3′ cyphers, a different cypher on each end, so that sequencing of each target nucleic acid molecule or portion thereof within a strand of tandem nucleic acid molecules produced by rolling circle amplification, and on a complementary strand, can be connected or linked back to the original molecule. The unique cypher flanking the target nucleic acid molecules or portions thereof on each rolling circle amplification strand links each target nucleic acid molecule or portion thereof with each other and with the original complementary strand (e.g., before any amplification), so that each linked sequence serves as its own internal control. In other words, by uniquely tagging double-stranded nucleic acid molecules, sequence data obtained from one strand of tandem repeats of a single nucleic acid molecule can be compared within a strand and specifically linked to sequence data obtained from the complementary strand of that same double-stranded nucleic acid molecule. Furthermore, sequence data obtained from one end of a double-stranded target nucleic acid molecule can be specifically linked to sequence data obtained from the opposite end of that same double-stranded target nucleic acid molecule (if, for example, it is not possible to obtain sequence data across the entire double-stranded nucleic acid molecule of the library). Compositions relating to double stranded nucleic acid molecule libraries comprising a plurality of nucleic acid molecules and a plurality of random cyphers, or a plurality of nucleic acid vectors comprising a plurality of random cyphers, or methods of use have been described in PCT Application titled “Compositions and Methods for Accurately Identifying Mutations,” serial number PCT/US2013/026505, filed on Feb. 15, 2013, which is hereby incorporated by reference in its entirety.

As used herein, “rolling circle amplification” or “rolling circle replication” or “rolling circle synthesis” refers an isothermal amplification method that utilizes a circular template for synthesizing multiple copies of nucleic acid molecules. During rolling circle amplification, a replication fork proceeds around a circular template for an indefinite number of revolutions. The nucleic acid strand newly synthesized in each revolution displaces the strand synthesized in the previous revolution, which is “rolled off” of the circular template, giving a tail containing linear series of sequences complementary to the circular template strand, also called a “concatemer” or “tandem nucleic acid molecules.” Rolling circle amplification techniques include methods that use circularized target nucleic acid molecules as template or methods that use circularized probes for interrogating linear target nucleic acid molecules. Rolling circle amplification includes using either a sense or anti-sense primer for unidirectional strand synthesis or both sense and anti-sense primers for bi-directional synthesis of complementary strands.

As used herein, a “nucleic acid molecule priming site” or “PS” and variants thereof are short, known nucleic acid sequences contained in the vector. A PS sequence can vary in length from 5 nucleotides to about 50 nucleotides in length, about 10 nucleotides to about 30 nucleotides, and preferably are about 15 nucleotides to about 20 nucleotides in length. In certain embodiments, a PS sequence may be included at the one or both ends or be an integral part of the random cypher nucleic acid molecules, or be included at the one or both ends or be an integral part of an adapter sequence, or be included as part of the vector. A nucleic acid molecule primer that is complementary to a PS included in a library of the present disclosure can be used to initiate a sequencing reaction.

For example, if a random cypher only has a PS upstream (5′) of the cypher, then a primer complementary to the PS can be used to prime a sequencing reaction to obtain the sequence of the random cypher and some sequence of a target nucleic acid molecule cloned downstream of the cypher. In another example, if a random cypher has a first PS upstream (5′) and a second PS downstream (3′) of the cypher, then a primer complementary to the first PS can be used to prime a sequencing reaction to obtain the sequence of the random cypher, the second PS and some sequence of a target nucleic acid molecule cloned downstream of the second PS. In contrast, a primer complementary to the second PS can be used to prime a sequencing reaction to directly obtain the sequence of the target nucleic acid molecule cloned downstream of the second PS. In this latter case, more target molecule sequence information will be obtained since the sequencing reaction beginning from the second PS can extend further into the target molecule than does the reaction having to extend through both the cypher and the target molecule.

As used herein, an “adapter” or “adapter sequence” refers to a sequence located upstream of the 5′ cypher or downstream of the 3′ cypher, or both, with a length ranging from about 20 nucleotides to about 100 nucleotides. Adapter sequences may contain sequences useful for amplification, sequencing, or other processing of the target nucleic acid molecules following rolling circle amplification. Adapter sequences may contain restriction endonuclease sites; or primer sites for bridge amplification, PCR amplification, or sequencing.

As used herein, “next generation sequencing” refers to high-throughput sequencing methods that allow the sequencing of thousands or millions of molecules in parallel. Examples of next generation sequencing methods include sequencing by synthesis, sequencing by ligation, sequencing by hybridization, polony sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.”

As used herein, “single molecule sequencing” or “third generation sequencing” refers to high-throughput sequencing methods wherein reads from single molecule sequencing instruments represent sequencing of a single molecule of DNA. Unlike next generation sequencing methods that rely on PCR to grow clusters of a given DNA template, attaching the clusters of DNA templates to a solid surface that is then imaged as the clusters are sequenced by synthesis in a phased approach, single molecule sequencing interrogates single molecules of DNA and does not require PCR amplification or synchronization. Single molecule sequencing includes methods that need to pause the sequencing reaction after each base incorporation (‘wash-and-scan’ cycle) and methods which do not need to halt between read steps. Examples of single molecule sequencing methods include single molecule real-time sequencing, nanopore-based sequencing, and direct imaging of DNA using advanced microscopy.

In certain embodiments, the present disclosure provides a method of detecting mutations in a target nucleic acid molecule, the method comprising: a) a first amplification step comprising rolling circle amplification of a library of double-stranded circular bar-coded template molecules with a first sense primer and a first anti-sense primer specific for a first target nucleic acid molecule, wherein the library of double-stranded circular bar-coded template molecules comprises vectors containing a plurality of double-stranded nucleic acid molecules, wherein each double-stranded nucleic acid molecule is flanked by a 5′ cypher and a 3′ cypher within the vector, wherein the 5′ cypher is different than the 3′ cypher for each double-stranded nucleic acid molecule, and wherein rolling circle amplification produces two complementary strands of tandem nucleic acid molecules comprising multiple copies of first target nucleic acid molecule or portion thereof; b) a second amplification step comprising amplification of the first target nucleic acid molecules or portions thereof and flanking 5′ and 3′ cyphers on each strand of tandem nucleic acid molecules produced from step a); and c) sequencing the first target nucleic acid molecules or portions thereof produced from step b), thereby detecting mutations in the first target nucleic acid molecule compared to a reference first target nucleic acid molecule sequence.

A target nucleic acid molecule is any nucleic acid molecule, including genomic DNA or mitochondrial DNA, in which detection of a mutation is desirable. In certain embodiments, a nucleic acid molecule is genomic DNA. In other embodiments, a nucleic acid molecule is mitochondrial DNA. A reference target nucleic acid molecule sequence is a wild type or normal sequence of a selected target nucleic acid molecule. A target nucleic acid molecule may have more than one reference sequence. Methods for isolating nucleic acid molecules for use in the methods described herein are well known in the art.

In certain embodiments, a mutation is a deletion of one or more nucleotides. In other embodiments, a mutation is an insertion or substitution of one or more nucleotides. A mutation may also include rearrangements of large segments of nucleotides, such as chromosomal translocations, inversions, or duplications. The disclosed methods can be used to detect any mutation within a target nucleic acid molecule.

A plurality of double-stranded nucleic acid molecules is cloned into vectors to form a library of double-stranded circular bar-coded template molecules. A “vector” is a nucleic acid molecule that is capable of transporting another nucleic acid. Vectors may be, for example, plasmids, cosmids, viruses, or phage. An “expression vector” is a vector that is capable of directing the expression of a protein encoded by one or more genes carried by the vector when it is present in the appropriate environment.

In certain embodiments, a plurality of nucleic acid molecules is obtained from a human subject. In other embodiments, a plurality of nucleic acid molecules is obtained from other subjects, including prokaryotic organisms, eukaryotic organisms, viruses, or viroids. Prokaryotic organisms include bacteria and archaea. Eukaryotic organisms include protozoa, algae, plants, slime molds, fungi (e.g., yeast), and animals. Animal organisms include mammals, such as primate, cow, dog, cat, rodent (e.g., mouse, rat, guinea pig), rabbit, or non-mammals, such as nematodes, bird, amphibian, reptile, or fish. A plurality of nucleic acid molecules can be from any sample from a subject, tissue or fluid, including a blood, tumor biopsy, tissue biopsy, saliva, sputum, cerebral spinal fluid, vaginal secretion, breast secretion, or urine. A sample may contain both normal and abnormal (diseased, infected, damaged, affected) tissue or cells. A sample can also be derived from a cell line. In certain embodiments, a plurality of nucleic acid molecules consists essentially of a single type of nucleic acid molecule, e.g., genomic DNA or mtDNA or mRNA. In other embodiments, a plurality of nucleic acid molecules consists essentially of more than one type of nucleic acid molecule, e.g., a mixture of genomic DNA and mtDNA. A plurality of nucleic acid molecules includes nucleic acid molecules from a variety of cells, tissues, organs, and sources within a subject, including diseased and normal tissues or wild type and mutant cells (e.g., circulating normal and tumor cells). A plurality of nucleic acid molecules may also be circulating as cell-free nucleic acid molecules, and extracted from plasma or other bodily fluids from a subject. A plurality of nucleic acid molecules can include nucleic acid molecules from more than one subject, such as nucleic acid molecules from mother and fetus or nucleic acid molecules from host and infectious agent (virus, bacteria, fungi, protozoa, parasite that causes an infectious disease or infection in the host).

Once isolated from a sample, a plurality of nucleic acid molecules may undergo further processing prior to cloning into vectors. Such processing includes mechanical shearing or cleavage with restriction endonucleases to generate shorter nucleic acid molecule fragments. Nucleic acid fragments having overhanging ends may be repaired (i.e., blunted) using T4 DNA polymerase and E. coli DNA polymerase I Klenow fragment. Ribonucleic acid molecules may undergo reverse transcription and cDNA synthesis to produce a plurality of double-stranded nucleic acid molecules for insertion into the vectors. A synthesis step may be performed on single stranded nucleic acid molecules to produce a plurality of double-stranded nucleic acid molecules for insertion into the vectors. A plurality of double-stranded nucleic acid molecules contained in the vectors range in size from about 10 nucleotides to several thousand nucleotides (e.g., 5,000). Preferably, the plurality of double-stranded nucleic acid molecules contained in the vectors range in size from about 50 nucleotides to about 3,000 nucleotides or from about 100 nucleotides to about 2,000 nucleotides, or from about 150 nucleotides to about 1,000 nucleotides. In certain embodiments, a plurality of double-stranded nucleic acid molecules range in size from about 100 to about 1,000 nucleotides, or from about 150 to about 750 nucleotides, or from about 250 nucleotides to about 500 nucleotides.

Within the vector, each double-stranded nucleic acid molecule is flanked by a 5′ cypher and a 3′ cypher, wherein the 5′ cypher is different than the 3′ cypher for each double-stranded nucleic acid molecule. A cypher or barcode is a double stranded nucleic acid sequence comprised of about 5 to about 50 nucleotides. In certain embodiments, all of the nucleotides of within a cypher are not identical (i.e., comprise at least two different nucleotides), and optionally do not contain three contiguous nucleotides that are identical. In further embodiments, the cypher is comprised of about 5 to about 15 nucleotides, preferably about 6 to about 10 nucleotides, and even more preferably 6, 7, or 8 nucleotides.

In further embodiments, the plurality or pool of random cyphers used in the double-stranded nucleic acid molecule library comprise from about 5 nucleotides to about 40 nucleotides, about 5 nucleotides to about 30 nucleotides, about 6 nucleotides to about 30 nucleotides, about 6 nucleotides to about 20 nucleotides, about 6 nucleotides to about 10 nucleotides, about 6 nucleotides to about 8 nucleotides, about 7 nucleotides to about 9 or about 10 nucleotides, or about 6, about 7 or about 8 nucleotides. In certain embodiments, the pair of unique random 5′ and 3′ cyphers associated with nucleic acid sequences will have different lengths or have the same length. For example, a double-stranded nucleic acid molecule may have a 5′ (upstream) cypher of about 6 nucleotides in length and a 3′ (downstream) cypher of about 9 nucleotides in length, or the double-stranded nucleic acid molecule may have an 5′ (upstream) cypher of about 7 nucleotides in length and a 3′ (downstream) cypher of about 7 nucleotides in length.

In certain embodiments, both the 5′ cypher and the 3′ cypher each comprise 6 nucleotides, 7 nucleotides, 8 nucleotides, 9 nucleotides, or 10 nucleotides. In certain embodiments, the 5′ cypher comprises 6 nucleotides and the 3′ cypher comprises 7 nucleotides or 8 nucleotides, or the 5′ cypher comprises 7 nucleotides and the 3′ cypher comprises 6 nucleotides or 8 nucleotides, or the 5′ cypher comprises 8 nucleotides and the 3′ cypher comprises 6 nucleotides or 7 nucleotides.

The number of nucleotides contained in each of the cyphers or bar codes will govern the total number of possible bar codes available for use in a library. Shorter bar codes allow for a smaller number of unique cyphers, which may be useful when performing a deep sequence of one or a few nucleotide sequences, whereas longer bar codes may be desirable when examining a population of nucleic acid molecules, such as cDNAs or genomic fragments. For example, a bar code of 7 nucleotides would have a formula of 5′-NNNNNNN-3′ (SEQ ID NO:1), wherein N may be any naturally occurring nucleotide. The four naturally occurring nucleotides are A, T, C, and G, so the total number of possible random cyphers is 4⁷, or 16,384 possible random arrangements (i.e., 16,384 different or unique cyphers). For 6 and 8 nucleotide bar codes, the number of random cyphers would be 4,096 and 65,536, respectively. In certain embodiments of 6, 7 or 8 random nucleotide cyphers, there may be fewer than the pool of 4,094, 16,384 or 65,536 unique cyphers, respectively, available for use when excluding, for example, sequences in which all the nucleotides are identical (e.g., all A or all T or all C or all G) or when excluding sequences in which three contiguous nucleotides are identical or when excluding both of these types of molecules. In addition, the first about 5 nucleotides to about 20 nucleotides of the target nucleic acid molecule sequence may be used as a further identifier tag together with the sequence of an associated random cypher.

For example, if the length of the random cypher is 7 nucleotides, then there will a total of 16,384 different bar codes available as first random 5′ cypher and second random 3′ cypher. In this case, if a first double-stranded nucleic acid molecule is associated with and disposed between random 5′ cypher number 1 and random 3′ cypher number 2, and a second double-stranded nucleic acid molecule is associated with and disposed between random 5′ cypher number 16,383 and random 3′ cypher number 16,384, then a third double-stranded nucleic acid molecule can only be associated with and disposed between any pair of random 5′ and 3′ cypher numbers selected from numbers 3-16,382, and so on for each double-stranded nucleic acid molecule of a library until each of the different random cyphers have been used (which may or may not be all 16,382). In this embodiment, each double-stranded nucleic acid molecule of a library will have a unique pair of 5′ and 3′ cyphers that differ from each of the other pairs of 5′ and 3′ cyphers found associated with each of the other double-stranded nucleic acid molecule of the library.

In certain embodiments, random cypher sequences from a particular pool of cyphers (e.g., pools of 4,094, 16,384 or 65,536 unique cyphers) may be used more than once provided that each double-stranded nucleic acid molecule has a different (unique) pair of 5′ and 3′ cyphers. For example, if a first double-stranded nucleic acid molecule is associated with and disposed between random 5′ cypher number 1 and random 3′ cypher number 100, then a second double-stranded nucleic acid molecule will need to be flanked by a different dual pair of cyphers—such as random 5′ cypher number 1 and random 3′ cypher number 65, or random 5′ cypher number 486 and random 3′ cypher number 100—which may be any combination other than 1 and 100.

In certain embodiments, double-stranded nucleic acid molecules of the library will each have dual unique 5′ and 3′ cyphers, wherein none of the 5′ cyphers have the same sequence as any other 5′ cypher, none of the 3′ cyphers have the same sequence as any other 3′ cypher, and none of the 5′ cyphers have the same sequence as any 3′ cypher. In still further embodiments, double-stranded nucleic acid molecules of the library will each have a unique pair of 5′-3′ cyphers wherein none of the 5′ or 3′ cyphers have the same sequence.

In still further embodiments, the plurality of random 5′ and 3′ cyphers may further comprise a nucleic acid molecule priming site upstream or downstream of the 5′ barcode sequence or upstream or downstream of the 3′ barcode sequence. In certain embodiments, a plurality of random cyphers may each be associated with and disposed between a first nucleic acid molecule priming site (PS1) and a second nucleic acid molecule priming site (PS2), wherein the double-stranded sequence of PS1 is different from the double-stranded sequence of PS2. In certain embodiments, each unique pair of 5′-3′ cyphers may be associated with and disposed between an upstream and a downstream first nucleic acid molecule priming site (PS1). In further embodiments, each unique pair of 5′-3′ cyphers may be associated with and disposed between two or more upstream and downstream nucleic acid molecule priming sites. Nucleic acid molecule priming sites upstream of the 5′ cypher and downstream of the 3′ cypher can be used for subsequent amplification and sequencing of the 5′ cypher—double stranded nucleic acid molecule—3′ cypher disposed within. By locating a priming site upstream of the 5′ cypher and a priming site downstream of the 3′ cypher, the barcode sequence may be associated with the double stranded nucleic acid molecule vector insert sequence in subsequent amplification and sequencing reactions.

In further embodiments, a first nucleic acid molecule priming site PS1 will be located upstream (5′) of the first random 5′ cypher and the first nucleic acid molecule priming site PS1 will also be located downstream (3′) of the second random 3′ cypher. In certain embodiments, an oligonucleotide primer complementary to the sense strand of PS1 can be used to prime a sequencing reaction to obtain the sequence of the sense strand of the first random 5′ cypher or to prime a sequencing reaction to obtain the sequence of the anti-sense strand of the second random 3′ cypher, whereas an oligonucleotide primer complementary to the anti-sense strand of PS1 can be used to prime a sequencing reaction to obtain the sequence of the anti-sense strand of the first random 5′ cypher or to prime a sequencing reaction to obtain the sequence of the sense strand of the second random cypher 3′.

In further embodiments, the second nucleic acid molecule priming site PS2 will be located downstream (3′) of the first random 5′ cypher and the second nucleic acid molecule priming site PS2 will also be located upstream (5′) of the second random 3′ cypher. In certain embodiments, an oligonucleotide primer complementary to the sense strand of PS2 can be used to prime a sequencing reaction to obtain the sequence of the sense strand from the 5′-end of the associated double-stranded target nucleic acid molecule or to prime a sequencing reaction to obtain the sequence of the anti-sense strand from the 3′-end of the associated double-stranded target nucleic acid molecule, whereas an oligonucleotide primer complementary to the anti-sense strand of PS2 can be used to prime a sequencing reaction to obtain the sequence of the anti-sense strand from the 5′-end of the associated double-stranded target nucleic acid molecule or to prime a sequencing reaction to obtain the sequence of the sense strand from the 3′-end of the associated double-stranded target nucleic acid molecule.

In certain embodiments, a plurality of random 5′ and 3′ cyphers further comprises a restriction endonuclease site. In additional embodiments, a plurality of random 5′ and 3′ cyphers further comprises a unique index sequence (comprising a length ranging from about 4 nucleotides to about 25 nucleotides) specific for a particular sample so that a library can be pooled with other libraries having different index sequences to facilitate multiplex sequencing (also referred to as multiplexing). In further embodiments a plurality of random 5′ and 3′ cyphers further comprises an adapter sequence comprising a length ranging from about 20 nucleotides to about 100 nucleotides, such adapter sequences may be used for bridge amplification.

The 5′ and 3′ cyphers may be ligated onto the plurality of double-stranded nucleic acid molecules prior to cloning into vectors. In a preferred embodiment, a vector library is constructed comprising a plurality of random 5′ and 3′ cyphers, into which the double-stranded nucleic acid molecules are cloned.

Dual random 5′ and 3′ cyphers, double stranded nucleic acid molecule libraries comprising a plurality of nucleic acid molecules and a plurality of random cyphers, nucleic acid vector libraries comprising a plurality of random cyphers, and methods of use have been previously described in PCT Application titled “Compositions and Methods for Accurately Identifying Mutations,” PCT Application No. PCT/US2013/026505, filed on Feb. 15, 2013, which is hereby incorporated by reference in its entirety.

A library of double-stranded circular bar-coded template molecules comprising vectors containing a plurality of double-stranded nucleic acid molecules is template for a first amplification step comprising rolling circle amplification. At least one primer (sense or antisense) specific for a first target nucleic acid molecule is selected for priming rolling circle amplification. In certain embodiments, a first sense primer and a first antisense primer specific for a first target nucleic acid molecule are used to prime rolling circle amplification. In some embodiments, a plurality of sense primers or a plurality of antisense primers, or a plurality of sense and antisense primers specific for a first target nucleic acid molecule is used for priming rolling circle amplification. In certain embodiments, at least 1, 2, 3, 4, 5, 6, 7, 8, 9, 10, 15, 20, 25, 30, 40, 50, 60, 70, 80, 90, to about 100 primers specific for a target nucleic acid molecule are used for the first amplification step. The number of primers specific for a target nucleic acid molecule may all comprise sense primers, may all comprise antisense primers, or may be evenly (e.g., 50 sense and 50 antisense) or unevenly (e.g., 49 sense and 51 antisense; 40 sense and 60 antisense; 30 sense and 70 antisense; 20 sense and 80 antisense; 10 sense and 90 antisense; 5 sense and 95 antisense; or any combination thereof) divided between sense and antisense primers.

A sense primer specific for a first target nucleic acid molecule can be used to anneal to the antisense strand of the target nucleic acid molecule and prime extension of the sense strand. An antisense primer specific for a first target nucleic acid molecule can be used to anneal to the sense strand of the target nucleic acid molecule and prime extension of the antisense strand. A pair of sense and antisense primers specific for a first target nucleic acid molecule can be used to anneal to the antisense and sense strands, respectively, of the target nucleic acid molecule and primer extension of the sense and antisense strands.

Primers specific for a first target nucleic acid molecule may be designed to amplify a selected region within a nucleic acid molecule (e.g., a mutational hot spot, an exon, an exon/intron boundary, a gene fragment) or multiple regions within a nucleic acid molecule, or designed to amplify an entire nucleic molecule. Primers specific for a first target nucleic acid molecule may be spaced from about 30, 40, 50, 60, 70, 80, 90, 100, 200, 300, 400, 500, 600, 700, 800, 900, 1,000, 1,500, or 2,000 nucleotides apart on the same strand of a first target nucleic acid molecule (e.g., sense primers are spaced from about 50 nucleotides apart). In certain embodiments, primers specific for a first target nucleic acid molecule are spaced from about 50 to about 1,000 nucleotides apart on the same strand a first target nucleic acid molecule. By utilizing a plurality of primers designed with selective positioning and spacing, entire nucleic acid molecules (e.g., genes, transcripts, genomes) may be interrogated in a single assay.

In certain embodiments, primers specific for a first target nucleic acid molecule further comprise nucleotides specific for the cypher or a portion thereof.

In certain embodiments, rolling circle amplification comprises at least one or more sense, antisense, or a combination thereof, primers specific for at least a second target nucleic acid molecule. In further embodiments, a plurality of sense, a plurality of antisense, or a combination thereof, primers specific for a plurality of different target nucleic acid molecules are used in rolling circle amplification, allowing multiplex detection of mutations in multiple target nucleic acid molecules. Methods described herein may be used to detect mutations in at least 1, 2, 3, 4, 5, 10, 15, 20, 25, 30, 35, 40, 45, 50, 60, 70, 80, 90, or 100 target nucleic acid molecules. In certain embodiments, about 10 primers specific for each target nucleic acid molecule, for up to 100 different target nucleic acid molecules (e.g., 1,000 primers total used to interrogate 100 different target nucleic acid molecules), are used in the first amplification step comprising rolling circle amplification.

In certain embodiments, a primer that is specific for a target nucleic acid molecule and used to prime rolling circle amplification is exonuclease resistant. Proofreading DNA polymerases, such as Klenow fragment, VENT® DNA polymerase, Pfu DNA polymerase, T7 DNA polymerase, and Φ29 DNA polymerase, have enhanced fidelities during amplification of DNA sequences by PCR. However, proofreading DNA polymerases also have 3′→5′ exonuclease activity that degrade the oligodeoxynucleotide primers needed for DNA synthesis. These shortened primer molecules may still be able to anneal to the template, but at lower temperatures and with reduced specificity. If the primers have been modified such that the 5′ terminal sequence does not match the template (e.g., to introduce restriction sites for cloning purposes or to add flanking nucleotides), then degraded primers are unlikely to give rise to an amplification product.

Exonuclease resistant oligonucleotide primers are known in the art. A exonuclease resistant primer may comprise an alkyl phosphonate monomer, RU P(═O)(-Me)(-OR), such as dA-Me-phosphonamidite, and/or a triester monomer, RO—P(═O)(—OR′)(—OR), such as dA-Me-phosphoramidite (available from Glen Research, Sterling, Va.), and/or a locked nucleic acid monomer (available from Exiqon, Woburn, Mass. and/or a boranophosphate monomer, RO—P(—BH₃)(═O)(—OR). Variation of the phosphate backbone is known in the art to provide exonuclease resistance (see, U.S. Pat. No. 5,256,775; PCT Publication WO89/05358; Dean et al., 2001, Genome Res. 11:1095-1099). In certain embodiments, a primer may comprise a phosphorothioate (PTO) modification (or two, three, or four or more phosphorothioate modifications) at its 3′ terminus. For example, a primer with a one phosphorothioate modification at its 3′ terminus has a phosphorothioate bond between the two terminal 3′ bases of the primer. A primer with two phosphorothioate modifications at its 3′ terminus has a phosphorothioate bond between the two terminal 3′ bases and between the 2^{nd} and 3^{rd} base upstream from the 3′ terminus.

A library of double stranded circular barcoded template molecules is amplified by rolling circle amplification, wherein a primer specific for a target nucleic acid molecule anneals to the circular or circularized target and undergoes numerous rounds of isothermal polymerase based extension of the hybridized primer by continuously progressing around the same circular template molecule. Rolling circle amplification methods are adapted from rolling circle replication used by many plasmids and viruses (Gilbert & Dressler, 1968, Cold Spring Harbor Symp. Quant. Biol. 33:473-484; Baker & Kornberg, 1991, DNA Replication, Freeman, New York). Rolling circle amplification methods have been previously described and include linear rolling circle amplification or hyper-branched rolling circle amplification (e.g., U.S. 5,648,245; Fire and Xu, 1995, Proc. Acad. Sci. USA 92:4641-4645; Liu et al., 1996, J. Am. Chem. Soc. 118:1587-1594; Lizardi et al., 1998, Nat. Genet. 19:225-232; Zhang et al., 1998, Gene 211:277-285). Rolling circle amplification may also use circularized probes to hybridize to linear template molecules (e.g., padlock probes) (Nilsson et al., 1994, Science 265:2085-2088).

From a sense primer specific for a target nucleic acid molecule, rolling circle amplification produces a strand of tandem nucleic acid molecules, which are complementary to the antisense sequence of the double-stranded circular bar-coded template molecule. The strand of tandem nucleic acid molecules comprises multiple copies of the target nucleic acid molecule or portion thereof. Rolling circle amplification may produce incomplete copies of the target nucleic acid molecule, particularly at the 3′ terminus of the strand. From an antisense primer specific for a target nucleic acid molecule, rolling circle amplification produces a strand of tandem nucleic acid molecules, which are complementary to the sense sequence of the double-stranded circular bar-coded template molecule. The strand of tandem nucleic acid molecules comprises multiple copies of the target nucleic acid molecule or portion thereof. If both a sense and an antisense primer specific for a target nucleic acid molecule are used in rolling circle amplification, bi-directional synthesis results in two strands of tandem nucleic acid molecules comprising multiple copies of first target nucleic acid molecule or portion thereof that are complementary to each other. If a plurality of sense (or antisense) primers specific for a target nucleic acid molecule is used, multiple strands of tandem nucleic acid molecules comprising multiple copies of first target nucleic acid molecule or portion thereof are produced. These multiple strands may be branching off the same circular template molecule simultaneously. The products of rolling circle amplification may further comprise one or more sequences for other components present within the double-stranded circular bar-coded template molecule, including vector sequence, 5′ and 3′ cyphers, priming sites, adapter sequences, restriction sites, or index sequences, arranged in linear repeats.

In certain embodiments, a first sense primer and a first anti-sense primer specific for a first target nucleic acid molecule each primer further comprising a “tag molecule.” In certain embodiments, a plurality of sense and antisense primers specific for a plurality of different target nucleic acid molecules each further comprise a tag molecule. A tag, or affinity tag, comprises a detectable molecule (biological or chemical) that allows for isolation or selection of its partner molecule to which the tag is attached (e.g., the products of target-specific primer-directed rolling circle amplification) via interactions with a binding substrate for the tag. A tag allows for isolation or selection that is independent of the tag's partner molecule's structure or sequence. Tag molecules may be attached using genetic methods or chemically coupled. Tag molecules are well known in the art and include, e.g., biotin, HIS tag, Flag® epitope, GST, chitin binding protein, and maltose binding protein. In certain embodiments, the tag molecule is biotin. In further embodiments, following rolling circle amplification, biotin-tagged strands of tandem nucleic acid molecules comprising multiple copies of first target nucleic acid molecule or portion thereof are selected or isolated with streptavidin or avidin before the second amplification step. In further embodiments, methods described herein can be repeated with the library of double-stranded circular bar-coded template molecules that have been purified to remove the biotin-tagged strands of tandem nucleic acid molecules.

A second amplification step (e.g., PCR) is performed comprising amplification of the first nucleic acid molecules, or portions thereof, and the flanking 5′ and 3′ cyphers on each strand of tandem nucleic acid molecules produced from rolling circle amplification. The second amplification step can selectively exclude undesirable sequence (e.g., vector sequence) for a subsequent sequencing step. The second amplification step can convert single strands of tandem nucleic acid molecules produced from rolling circle amplification into double stranded DNA for a subsequent sequencing step. In certain embodiments, primers specific for adapter sequences associated with the cyphers, priming sites associated with the cyphers, index sequence associated with the cyphers, or vector sequence upstream and downstream from the 5′ and 3′ cyphers and intervening target nucleic acid molecule may be used for the second amplification step. In further embodiments, priming sites associated with the cyphers are designed such that primers specific for the priming sites can be used for the second amplification step and/or for sequencing. In some embodiments, the same primer set (e.g., primers specific for vector sequence, priming sites, or adapter sequences present throughout the library) may be used for the second amplification step to amplify multiple target nucleic acid molecules or portions thereof produced from a multiplex rolling circle amplification reaction. In certain embodiments, the primers are be designed to contain sequence specific for 5′ and 3′ cyphers.

In further embodiments, first target nucleic acid molecules, or portions thereof, produced from the second amplification step are sequenced, thereby detecting mutations in the first target nucleic acid molecule as compared to a reference first target nucleic acid molecule sequence. A variety of sequencing methods known in the art, such as sequencing by synthesis, pyrosequencing, reversible dye-terminator sequencing, polony sequencing, or single molecule sequencing may be used.

Depending on the length of the target nucleic acid molecule, the entire nucleic acid molecule sequence may be obtained (e.g., if less than about 100 nucleotides to about 250 nucleotides if this is the limit for the particular sequencing technique used) or only a portion of the entire target nucleic acid molecule sequence may be obtained (e.g., about 100 nucleotides to about 250 nucleotides if this is the limit for the particular sequencing technique used). An advantage of the compositions and methods of the present disclosure is that even though a target nucleic acid molecule may be too long to obtain sequence data for the entire molecule or fragment, the sequence data obtained from one end of a double-stranded target nucleic molecule can be specifically linked to sequence data obtained from the opposite end of that same double-stranded target nucleic molecule because each nucleic molecule in a library of this disclosure will have a dual unique 5′ and 3′ cyphers, or a unique 5′-3′ pair of cyphers.

In certain embodiments, the sequencing step further comprises aligning the sequences of each first target nucleic acid molecule or portion thereof from one strand of tandem nucleic acid molecules (produced from rolling circle amplification) with each other. For example, each copy of first target nucleic acid molecule or portion thereof, present on a strand (or multiple same directional strands) produced by rolling circle amplification can be identified by its unique 5′ and 3′ cyphers. These sequences may be aligned, and a mutation may be distinguished as a polymerase error artifact or a true mutation by a person of skill in the art. Since rolling circle amplification uses the same circular template for each round of replication, a true mutation in a target nucleic acid molecule is likely to be present in all of the copies present on all same directional strands produced from the same template molecule, which may be identified by their unique 5′ and 3′ cyphers. Such comparison of all the copies of the first target nucleic acid molecule or portion thereof, present on a strand (or multiple same directional strands) may reduce the error rate to about 10⁻⁴ to about 10⁻⁵ or less.

In further embodiments, the sequencing step further comprises aligning the sequences of each first target nucleic acid molecule or portion thereof from one strand of tandem nucleic acid molecules (produced from rolling circle amplification) with each other and aligning with the sequences of each first target nucleic acid molecule or portion thereof from the complementary strand of tandem nucleic acid molecules (produced from rolling circle amplification). For example, each copy of first target nucleic acid molecule or portion thereof, present on complementary strands (including multiple sense and antisense strands) produced by rolling circle amplification can be identified by their unique 5′ and 3′ cyphers. These sequences may be aligned. A true mutation in a target nucleic acid molecule is likely to be present in all of the copies present on all same directional strands produced from the same template molecule, as well as on all complementary strands produced from the same template molecule, which may be identified by their unique 5′ and 3′ cyphers. Such comparison of all the copies of the first target nucleic acid molecule or portion thereof, present on complementary strands (sense and antisense) may reduce the error rate to at least below 10⁻⁶ to about 10⁻¹⁰ or less.

In certain embodiments, the sequencing step further comprises alignment of the sequences of each first target nucleic acid molecule or portion thereof from one strand of tandem nucleic acid molecules with each other and alignment with the sequences of each first target nucleic acid molecule or portions thereof from the complementary strand of tandem nucleic acid molecules, wherein the aligned sequences of each first target nucleic acid molecule or portion thereof from each strand of tandem nucleic acid molecules have matching 5′ and 3′ cyphers, and wherein the alignment results in a consensus sequence with a measureable sequencing error rate equal to or at least below 10⁻⁶ or less (e.g., 10⁻⁷, 10⁻⁸, 10⁻⁹, or 10⁻¹⁰ or less).

In certain embodiments, a plurality of target nucleic acid molecules, or portions thereof, produced from the second amplification step are sequenced, thereby detecting mutations in the plurality of target nucleic acid molecule as compared to reference target nucleic acid molecule sequences. Sequences of a plurality of target nucleic acid molecules, or portions thereof with matching 5′ and 3′ cyphers may also be aligned as described herein for sensitive and accurate detection of mutations.

In certain embodiments, the methods of this instant disclosure are useful for detecting rare mutants against a large background signal, such as for monitoring circulating tumor cells; detecting circulating mutant DNA in blood, detecting fetal DNA in maternal blood, monitoring or detecting disease and rare mutations by direct sequencing, monitoring or detecting disease or drug response-associated mutations. Additional embodiments may be used to quantify DNA damage or quantify or detect mutations in infectious agents (e.g., during HIV and other viral infections) that may be indicative of response to therapy or may be useful in monitoring disease progression or recurrence. In yet other embodiments, these compositions and methods are useful for detecting damage to DNA from chemotherapy, or for detecting and quantitating of specific methylation of DNA sequences.

For example, the methods described herein can be used to monitor mutational spectrum of tumor suppressor genes or oncogenes in a sample from a subject. Exemplary targets of interest are associated with one or more hyperproliferative disease, such as cancer, including, for example, BCR-ABL, RAS, RAF, MYC, P53, ER (Estrogen Receptor), HER2, EGFR, AKT, PI3K, mTOR, VEGF, ALK, pTEN, RB, DNMT3A, FLT3, NPM1, IDH1, IDH2, or the like. In certain embodiments, identification of certain target molecule mutations would reveal a population of subjects for which one or more medications (such as imatinib, vemurafenib, tamoxifen, toremifene, traztuzumab, lapatinib, cetuximab, panitumumab, rapamycin, temsirolimus, everolimus, vandetanib, bevacizumab, crizotinib) known to provide a therapeutic or prophylactic effect could be chosen for treatment of that specifically identified population of subjects, or are not chosen when it is known the one or more medications fails to provide a therapeutic or prophylactic effect to the specifically identified population of subjects.

Another aspect of the present application provides a method for enriching a target nucleic acid molecule over background level using rolling circle amplification. The method may be used to enrich a single target nucleic acid molecule or multiple target nucleic acid molecules from a mixed population of nucleic acid molecules. After enrichment, target nucleic acid molecules can be sequenced to detect mutations, polymorphisms, and the like.

In certain embodiments, the method for enriching a target nucleic acid molecule comprises: (a) a first amplification step comprising rolling circle amplification of a library of double-stranded circular bar-coded template molecules with a first sense or antisense primer specific for a first target nucleic acid molecule, wherein the library of double-stranded circular bar-coded template molecules comprises vectors containing a plurality of double-stranded nucleic acid molecules, wherein each double-stranded nucleic acid molecule is flanked by a 5′ cypher and a 3′ cypher within the vector, wherein the 5′ cypher is different than the 3′ cypher for each double stranded nucleic acid molecule, and wherein rolling circle amplification produces a strand of tandem nucleic acid molecules comprising multiple copies of the first target nucleic acid molecule or portion thereof, thereby enriching the target nucleic acid molecule.

In certain embodiments, a primer used to prime rolling circle amplification is an exonuclease resistant primer. In some embodiments, the primer comprises at least one, two, three, four, or more phosphothioate modified intersubunit linkages at its 3′ terminus.

In certain embodiments, the cyphers comprise a length ranging from about 5 nucleotides to about 10 nucleotides.

In certain embodiments, the cyphers further comprise a nucleic acid molecule priming site. In certain embodiments, the cyphers further comprise at least one adapter sequence.

In certain embodiments, the first primer further comprises a tag molecule. In some embodiments, the tag molecule is biotin. Tagged primer allows purification of rolling circle amplification product by using a substrate specific for the tag to isolate strands of tandem nucleic acid molecules comprising multiple copies of the first target nucleic acid molecule or portion thereof. Following the purification step, the library of double-stranded circular bar-coded template molecules can be re-used in another round of enrichment of a target nucleic acid molecule.

In certain embodiments, the plurality of double-stranded nucleic acid molecules is genomic DNA. In some embodiments, the plurality of double-stranded nucleic acid molecules is human. In some embodiments, the plurality of double-stranded nucleic acid molecules is obtained from a cell line, a tumor sample, a blood sample, or a biopsy sample.

In certain embodiments, the plurality of double-stranded nucleic acid molecules comprise a length ranging from about 100 to about 3,000 bases. In some embodiments, the plurality of double-stranded nucleic acid molecules contained in the vectors range in size from about 50 nucleotides to about 3,000 nucleotides, from about 100 nucleotides to about 2,000 nucleotides, from about 150 nucleotides to about 1,000 nucleotides, from about 100 to about 1,000 nucleotides, from about 150 to about 750 nucleotides, or from about 250 nucleotides to about 500 nucleotides.

In certain embodiments, the target nucleic acid molecule comprises an oncogene, tumor suppressor gene, or fragment thereof. In some embodiments, the tumor suppressor gene is TP53. In some embodiments, the target nucleic acid molecule is BCR-ABL, RAS, RAF, MYC, P53, ER (Estrogen Receptor), HER2, EGFR, AKT, PI3K, mTOR, VEGF, ALK, pTEN, RB, DNMT3A, FLT3, NPM1, IDH1, or IDH2.

In certain embodiments a target nucleic acid molecule is enriched at least 10², 10³, 10⁴, 10⁵, 10⁶, 10⁷, 10⁸, or 10⁹-fold over background levels.

In certain embodiments the rolling circle amplification step further comprises a second primer specific for a first target nucleic acid molecule, wherein rolling circle amplification produces two strands of tandem nucleic acid molecules comprising multiple copies of the first target nucleic acid molecule or portion thereof. The second primer can have the same direction as the first primer (both sense or both antisense), resulting in two same directional strands of tandem nucleic acid molecules comprising multiple copies of the first target nucleic acid molecule or portion thereof. The second primer can be antisense to the first sense or can be sense to the first antisense primer, such that rolling circle amplification produces two complementary strands of tandem nucleic acid molecules comprising multiple copies of the first target nucleic acid molecule or portion thereof. In some embodiments, the rolling circle amplification step further comprises 3, 4, 5, 6, 7, 8, 9, 10, 15, 20, 25, 30, 35, 40, 45, 50, 55, 60, 75, 80, 90, 100 or more primers specific for a first target nucleic acid molecule. In certain embodiments, the method further comprises rolling circle amplification with a plurality of primers specific for a plurality of different target nucleic acid molecules for a multiplexed reaction.

In certain embodiments, the method further comprises following the rolling circle amplification step, a second amplification step comprising amplification of the first target nucleic acid molecules or portions thereof and flanking 5′ and 3′ cyphers on each strand of tandem nucleic acid molecules produced from step (a); and sequencing the first target nucleic acid molecules or portions thereof produced from step (b).

Any of the aforementioned aspects, descriptions, and embodiments of target nucleic acid molecules, plurality of double-stranded nucleic acid molecules, vectors, library of double-stranded circular bar-coded template molecules, primers, primer modifications, rolling circle amplification, cyphers, adapters, priming sites, index sequences, strand of tandem nucleic acid molecules comprising multiple copies of the target nucleic acid molecule, and sequencing methods described herein for the methods for detecting mutations can be used in various embodiments of the methods of enrichment.

### EXAMPLES

### Example 1

### Rolling Circle Amplification and Dual Cypher Sequencing of a Tumor Genomic Library

Cancer cells contain numerous clonal mutations, i.e., mutations that are present in most or all malignant cells of a tumor and have presumably been selected because they confer a proliferative advantage. An important question is whether cancer cells also contain a large number of random mutations, i.e., randomly distributed unselected mutations that occur in only one or a few cells of a tumor. Such random mutations could contribute to the morphologic and functional heterogeneity of cancers and include mutations that confer resistance to therapy. Distinguishing clonal mutations from random mutations

To examine whether malignant cells exhibit a mutator phenotype resulting in the generation of random mutations in genes that would confer chemotherapeutic drug resistance, rolling circle amplification and dual cypher sequencing of present disclosure will be performed on normal and tumor genomic libraries.

Briefly, genomic DNA from patient-matched normal and tumor tissue is prepared using QIAGEN® kits (Valencia, Calif.), and quantified by optical absorbance and quantitative PCR (qPCR). The isolated genomic DNA is fragmented to a size of about 150-250 base pairs (short insert library) or to a size of about 300-700 base pairs (long insert library) by shearing. The DNA fragments having overhang ends are repaired (i.e., blunted) using T4 DNA polymerase and E. coli DNA polymerase I Klenow fragment, and then purified. The end-repaired DNA fragments are then ligated into the SmaI site of the library of dual cypher vectors as described in PCT Application titled “Compositions and Methods for Accurately Identifying Mutations,” Application No. PCT/US2013/026505, filed on Feb. 15, 2013, to generate a target genomic library. The ligated cypher vector library is purified and the target genomic library fragments are amplified by using rolling circle amplification (RCA) with sense and antisense biotin linked primers that anneal to regions that flank catalogued drug resistance mutations in ER (tamoxifen, toremifene), HER2 (traztuzumab, lapatinib), EGFR (cetuximab, panitumumab), mTOR (temsirolimus, everolimus), VEGF (vandetanib, bevacizumab), and ALK (crizotinib). For preparation target enrichment, between 0.1 ng and 100 ng of ligated cypher vector library is incubated in an annealing buffer consisting of 100 μL of 20 mM Tris-HCl (pH7.5), 40 mM NaCl, 1 mM EDTA, and 50 pmol pUC19-specific primer(s). The sample is incubated at 72° C. for 5 minutes and then allowed to slow-cool to room temperature. All RCA samples reactions are performed in 20 μL of 1× phi29 DNA Polymerase Reaction Buffer (New England Biolabs) supplemented with 200 ug/mL Bovine Serum Albumin, 200 uM dNTPs, 0.02 U Yeast Inorganic Pyrophosphatase, and 1 U of phi 29 polymerase (New England Biolabs). Samples are incubated at 30° C. for the duration of the reaction, and then heat inactivated at 65° C. for 10 minutes to halt rolling circle amplification. Following rolling circle amplification, 20 μl of the biotinylated DNA fragments are resuspended with 50 μg prewashed Dynabeads M-280-Streptavidin and 20 μl Kilobase binding solution (Dynal Biotech) and incubated at room temperature for 3 h on a roller. The bead solution is then placed in the Dynal Magnetic Particle Concentrator (MPC) (Dynal Biotech) and the supernatant removed. The Dynabead-DNA complex is washed twice in 40 μl washing solution (10 mM Tris-HCl, 1 mM EDTA, 2.0 M NaCl) and resuspended in 50 μl of 10 mM Tris-HCl (pH 7.9). The sample is incubated at 100° C. for 5 min, immediately placed in the MPC, washed with 500 μl 1 M NaCl and resuspended in 100 μl 1 M NaCl. The purified amplicons are then subject to a second amplification step using PCR with primers that flank the dual cyphers; using for example, the following PCR protocol: 30 seconds at 98° C.; five to thirty cycles of 10 seconds at 98° C., 30 seconds at 65° C., 30 seconds at 72° C.; 5 minutes at 72° C.; and then store at 4° C. The amplification is performed using sense strand and anti-sense strand primers that anneal to a sequence located within the adapter region, which sequence is upstream of the AS (or is even a part of the AS sequence), the unique cypher, and the target genomic insert (and, if present, upstream of an index sequence if multiplex sequencing is desired) for Illumina bridge sequencing. The sequencing of the library described above will be performed using, for example, an Illumina® Genome Analyzer II sequencing instrument as specified by the manufacturer.

The unique cypher tags are used to computationally deconvolute the sequencing data and map all sequence reads to single molecules (i.e., distinguish PCR and sequencing errors from real mutations). Base calling and sequence alignment are performed using, for example, the Eland pipeline (Illumina, San Diego, Calif.). The data generated allows identification of tumor heterogeneity and drug resistance mutations with single-nucleotide resolution at an unprecedented sensitivity.

### Example 2

### Rolling Circle Amplification and Dual Cypher Sequencing of a mtDNA Library

Mutations in mitochondrial DNA (mtDNA) lead to a diverse collection of diseases that are challenging to diagnose and treat. Each human cell has hundreds to thousands of mitochondrial genomes and disease-associated mtDNA mutations are homoplasmic in nature, i.e., the identical mutation is present in a preponderance of mitochondria within a tissue (Taylor and Turnbull, Nat. Rev. Genet. 6:389, 2005; Chatterjee et al., Oncogene 25:4663, 2006). Although the precise mechanisms of mtDNA mutation accumulation in disease pathogenesis remain elusive, multiple homoplasmic mutations have been documented in colorectal, breast, cervical, ovarian, prostate, liver, and lung cancers (Copeland et al., Cancer Invest. 20:557, 2002; Brandon et al., Oncogene 25:4647, 2006). Hence, the mitochondrial genome provides excellent potential as a more specific biomarker of disease than any other yet described, which may allow for improved treatment outcomes and, thereby, increase overall survival.

Rolling circle amplification and dual cypher sequencing methods of present disclosure can be leveraged to quantify circulating tumor cells (CTCs), and circulating tumor mtDNA (ctmtDNA) could be used to diagnose and stage cancer, assess response to therapy, and evaluate progression and recurrence after surgery. First, mtDNA isolated from prostatic cancer and peripheral blood cells from the same patient will be sequenced to identify somatic homoplasmic mtDNA mutations. These mtDNA biomarkers will be statistically assessed for their potential fundamental and clinical significance with respect to Gleason score, clinical stage, recurrence, therapeutic response, and progression.

Once specific homoplasmic mutations from individual tumors are identified, patient-matched blood specimens are examined for the presence of identical mutations in the plasma and buffy coat to determine the frequencies of ctmtDNA and CTCs, respectfully. This is accomplished by using the rolling circle amplification and dual cypher sequencing technology of this disclosure, and as described in Example 1, to sensitively monitor multiple mtDNA mutations concurrently. The distribution of CTCs in peripheral blood from patients with varying PSA serum levels and Gleason scores is determined.

### Example 3

### Targeted Enrichment of Dual Cypher Library Molecules by Rolling Circle Amplification

High grade serous ovarian carcinoma (HGSC) frequently exhibit somatic TP53 mutations (Cancer Genome Atlas Research Network, Nature 474:609, 2011). Loss of p53 is associated with unfavorable outcome (Kobel et al., 2010, J. Pathol. 222:191-198). Thus, the frequency and clinical value of TP53 mutations in HGSC make TP53 a promising biomarker for early detection and disease monitoring of HGSC. Enrichment methods of the present disclosure were used to enrich TP53 exon 4, a region that is frequently mutated in cancer, from an ovarian cancer cell line.

CaOV (human ovarian carcinoma cell line) cells were grown in McCoy's 5a Medium supplemented with 10% Fetal Bovine Serum, 1.5 mM/L-glutamine, 2200 mg/L sodium bicarbonate, and Penicillin/Streptomycin. CaOV cells were harvested and DNA was extracted using a DNeasy Blood and Tissue Kit (Qiagen). A target genomic library was created containing whole genomic DNA from CaOV, randomly sheared into DNA fragments an average of 150 bp long. DNA fragments having overhang ends were repaired (i.e., blunted) using T4 DNA polymerase, and the 5′-ends of the blunted DNA were phosphorylated with T4 polynucleotide kinase (Quick Blunting Kit I, New England Biolabs), and then purified. The end-repaired DNA fragments were blunt-end ligated into the SmaI site of a library of dual cypher vectors. The vector insert site is flanked by unique double-stranded cyphers each of which comprises a random 7-nucleotide barcode. Library priming sequences located 5′ to the 5′ cypher and 3′ to the 3′ cypher were also included in the vector, to allow amplification of the vector library. By uniquely tagging double-stranded nucleic acid molecules with the dual cyphers, each nucleic acid molecule can be individually identified, and sequence data obtained from one strand of a single nucleic acid molecule can be specifically linked to sequence data obtained from the complementary strand of that same double-stranded nucleic acid molecule. Methods of constructing dual cypher vectors and CypherSEQ libraries are described in PCT Application No. PCT/US2013/026505 (herein incorporated by reference in its entirety).

In brief, rolling circle amplification (RCA) was performed on this library using φ29 polymerase and a 5′-biotinylated, phosphothioate-modified primer specific to p53 exon 4. A portion of each reaction volume was purified by magnetic streptavidin beads. RCA reactions, including no-template, no-primer, and no-polymerase controls, were measured via SYBR Green-based quantitative polymerase chain reaction (qPCR), with primers specific to a 63 bp region of p53 exon 4 and another primer set specific to RNaseP, as an off-target control. Additionally, the p53 exon 4 forward primer, which binds to the same bases as the p53 exon 4 RCA primer, was paired with either the forward or reverse CypherSEQ library primer to measure any amplified p53 exon 4 molecules that did not include the p53 exon 4 reverse primer binding site.

Genomic DNA from CaOV ovarian cancer cells was randomly sheared to ˜150 bp and integrated into the CypherSEQ library construct, as described previously. To enrich for molecules containing a region of interest in exon 4 of p53, rolling circle amplification (RCA) with a target-specific primer was performed on the library prior to massively parallel sequencing. The RCA primer was altered to include a 5′-biotin modification for downstream purification by magnetic streptavidin beads. Additionally, phosphothioate modifications were added to the oligo, in the two internucleotidic linkages between the three 3′ bases of the primer. These phosphothioate modifications are resistant to the 3′ to 5′ exonuclease activity of the Φ29 polymerase, prevent primer degradation, and improve rolling circle amplification by up to 10⁶-fold. First, 500 μg/μL of CaOV CypherSEQ library DNA was mixed in a denaturing buffer (40 mM NaCl, 1 mM EDTA, and 4 mM Tris-HCl pH 7.8) with 5 μM of the p53 exon 4 RCA primer (5′-Biotin-CTGCCCTCAACAAGATGTTT-3′ (SEQ ID NO:2)). Mixes without DNA and without RCA primer were included as controls. 20 μL RCA reactions were performed with 1 μL of the above mixture, 1×Φ29 polymerase buffer (New England Biolabs), 10 units Φ29 polymerase (New England Biolabs), 500 nM each dNTP, and 4 ng BSA. Controls lacking polymerase were also included. RCA reactions were incubated at 37° C. for 5 days. A portion of each reaction was subjected to a magnetic streptavidin bead purification with the Dynabeads® kilobaseBINDER™ Kit (Life Technologies), according to the vendor's recommended protocol.

Rolling circle amplification products containing p53 exon 4 are then prepared for next generation sequencing platforms (e.g., Illumina® Genome Analyzer II) as described in Example 1 or PCT Application No. PCT/US2013/026505. Wild-type TP53 exon 4 sequence is compared to the actual sequence results to detect diversity of mutations.

### Example 4

### Measurement of Rolling Circle Amplification by Quantitative PCR

The effectiveness and specificity of the RCA reactions were measured by quantitative PCR, with primers targeted to p53 exon 4 (FOR: 5′-CTGCCCTCAACAAGATGTTT-3′ (SEQ ID NO:3), REV: 5′-AATCAACCCACAGCTGCAC-3′ (SEQ ID NO:4)) or RPP30 as an off-target genomic control (FOR: 5′-AGATTTGGACCTGCGAGC-3′ (SEQ ID NO:5), REV: 5′-GAGCGGCTGTCTCCACAAGT-3′ (SEQ ID NO:6)). Due to the random shearing prior to library construction, there is a high likelihood that library molecules amplified by RCA would exclude the binding site for the p53 exon 4 reverse primer. To investigate the frequency of this occurrence, wells with the p53 exon 4 forward primer and one of two “library” primers (FOR: 5′-AATGATACGGCGACCACCGA-3′ (SEQ ID NO:7), REV: 5′-CAAGCAGAAGACGGCATACGA-3′ (SEQ ID NO:8)), which flank the insert site of the CypherSEQ construct, were included to measure every RCA product amplified by the p53 exon 4 RCA primer. qPCR wells contained 25 μL reaction volumes with 1× GoTaq HotStart Master Mix (Promega), a 1:50,000 dilution of SYBR Green I (Lonza), 500 nM of each primer, and appropriate dilutions of each RCA reaction. Reaction volumes were thermally cycled on a CFX96 Real-Time PCR Detection System (Bio-rad) with the following conditions: 95° C. for 10 minutes, 45 cycles of 95° C. for 30 seconds, 61° C. for 60 seconds, and 72° C. for 90 seconds, followed by 72° C. for 5 minutes. Quantification was performed on CFX Manager software (Bio-rad) using a comparative C(t) method.

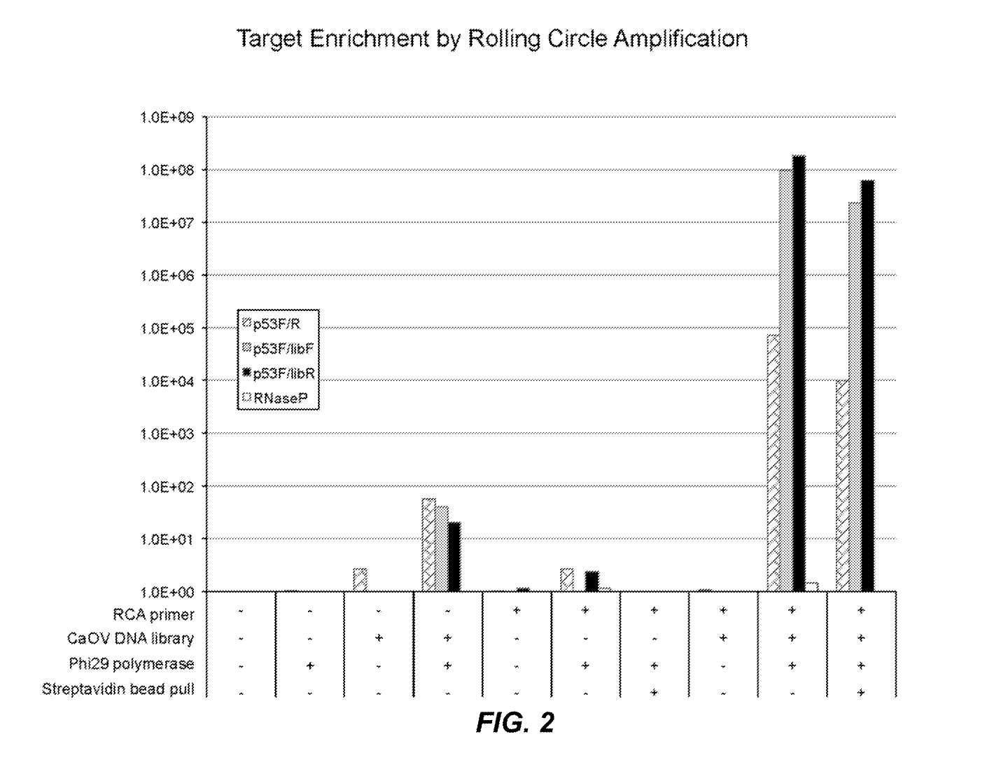

The results show nearly 10⁵-fold amplification or enrichment of the complete 63 bp region of p53 exon 4, and 10⁴-fold effective amplification after streptavidin bead purification (FIG. 2, hatched bar). Comparatively, qPCR with the p53 exon 4 forward and CypherSEQ library forward/reverse primer pairs displayed roughly 10⁸-fold and 10⁷-fold amplification pre- and post-bead purification, respectively (FIG. 2, gray and black bars). Only 1-2 copies of the RNaseP off-target control were detectable after RCA, and these were eliminated by bead purification (FIG. 2, white bar).

The various embodiments described herein can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method of detecting mutations in a target nucleic acid molecule, the method comprising:
(a) a first amplification step comprising rolling circle amplification of a library of double-stranded circular bar-coded template molecules with a first sense primer and a first anti-sense primer specific for a first target nucleic acid molecule,
wherein the library of double-stranded circular bar-coded template molecules comprises vectors containing a plurality of double-stranded nucleic acid molecules,
wherein each double-stranded nucleic acid molecule is flanked by a 5′ cypher and a 3′ cypher within the vector, wherein the 5′ cypher is different than the 3′ cypher for each double-stranded nucleic acid molecule, and
wherein rolling circle amplification produces two complementary strands of tandem nucleic acid molecules comprising multiple copies of the first target nucleic acid molecule or portion thereof;
(b) a second amplification step comprising amplification of the first target nucleic acid molecules or portions thereof and flanking 5′ and 3′ cyphers on each strand of tandem nucleic acid molecules produced from step a); and
(c) sequencing the first target nucleic acid molecules or portions thereof produced from step b), thereby detecting mutations in the first target nucleic acid molecule compared to a reference first target nucleic acid molecule sequence.

2. The method of claim 1, wherein the plurality of double-stranded nucleic acid molecules is genomic DNA or mitochondrial DNA.

3. The method of claim 1, wherein the plurality of double-stranded nucleic acid molecules is human.

4. The method of claim 1, wherein the plurality of double-stranded nucleic acid molecules is obtained from a tumor sample, a blood sample, or a biopsy sample.

5. The method of claim 1, wherein the plurality of double-stranded nucleic acid molecules comprises a length ranging from about 15 to about 3,000 base pairs.

6. The method of claim 1, wherein the cyphers comprise a length ranging from about 5 nucleotides to about 50 nucleotides.

7. The method of claim 1, wherein the cyphers comprise a length ranging from about 5 nucleotides to about 10 nucleotides or a length ranging from about 5 nucleotides to about 8 nucleotides.

8. The method of claim 1, wherein the cyphers further comprise a nucleic acid molecule priming site.

9. The method of claim 1, wherein the cyphers further comprise at least one adapter sequence.

10. The method of claim 1, wherein the first sense primer or first antisense primer specific for the first target nucleic acid molecule further comprises nucleotides specific for the cypher or a portion thereof.

11. The method of claim 1, wherein the first amplification step further comprises a second sense primer and a second anti-sense primer specific for the first target nucleic acid molecule.

12. The method of claim 7, wherein the first amplification step further comprises a plurality of sense primers and a plurality of antisense primers specific for the first target nucleic acid molecule.

13. The method of claim 1, wherein:
step a) further comprises amplifying by rolling circle amplification the double-stranded circular template molecules with a first sense primer and a first antisense primer specific for a second target nucleic acid molecule, wherein rolling circle amplification produces two complementary strands of tandem nucleic acid molecules comprising multiple copies of second target nucleic acid molecule or portion thereof;
step b) further comprises amplifying the second target nucleic acid molecules or portions thereof and flanking 5′ and 3′ cyphers on each strand of tandem nucleic acid molecules produced from step a); and
step c) further comprises sequencing the second target nucleic acid molecules or portions thereof produced from step b), thereby detecting mutations in the second target nucleic acid molecule compared to a reference second target nucleic acid molecule sequence

14. The method of claim 13, wherein the first amplification step further comprises a second sense primer and a second anti-sense primer specific for the second target nucleic acid molecule.

15. The method of claim 1, wherein the method comprises amplifying with a plurality of sense and antisense primers specific for a plurality of different target nucleic acid molecules.

16. The method of claim 15, wherein a plurality of different target nucleic acid molecules is about 2 to about 100 different target nucleic acid molecules.

17. The method of claim 8 or 9, wherein the first target nucleic acid molecules or portions thereof produced from step a) are amplified with primers specific for the priming site or adapter sequence.

18. The method of claim 1, wherein the sequencing is sequencing by synthesis, pyrosequencing, reversible dye-terminator sequencing, polony sequencing, or single molecule sequencing.

19. The method of claim 1, wherein the sequencing step further comprises alignment of the sequences of each first target nucleic acid molecule or portion thereof from one strand of tandem nucleic acid molecules with each other and alignment with the sequences of each first target nucleic acid molecule or portions thereof from the complementary strand of tandem nucleic acid molecules,
wherein the aligned sequences of each first target nucleic acid molecule or portion thereof from each strand of tandem nucleic acid molecules have matching 5′ and 3′ cyphers, and
wherein the alignment results in a consensus sequence with a measureable sequencing error rate equal to or at least below 10⁻⁶.

20. The method of claim 1, wherein the first target nucleic acid molecule is p53.

21. The method of claim 15, wherein the plurality of different target nucleic acid molecules comprise tumor suppressor genes or oncogenes.

22. The method of claim 1, wherein the first sense primer and the first anti-sense primer specific for the first target nucleic acid molecule each further comprises a tag molecule.

23. The method of claim 22, wherein the tag molecule is biotin.

24. The method of claim 22, wherein the method further comprises:
selection of the two complementary strands of tandem nucleic acid molecules comprising multiple copies of first target nucleic acid molecule or portion thereof with streptavidin or avidin following step a) and before step b).

25. The method of claim 24, wherein the method can be repeated with the library of double-stranded circular barcoded template molecules after selection with streptavidin or avidin.

26. A method of enriching a target nucleic acid molecule comprising:
(a) a first amplification step comprising rolling circle amplification of a library of double-stranded circular bar-coded template molecules with a first sense or antisense primer specific for a first target nucleic acid molecule,
wherein the library of double-stranded circular bar-coded template molecules comprises vectors containing a plurality of double-stranded nucleic acid molecules,
wherein each double-stranded nucleic acid molecule is flanked by a 5′ cypher and a 3′ cypher within the vector, wherein the 5′ cypher is different than the 3′ cypher for each double stranded nucleic acid molecule, and
wherein rolling circle amplification produces a strand of tandem nucleic acid molecules comprising multiple copies of the first target nucleic acid molecule or portion thereof, thereby enriching the target nucleic acid molecule.

27. The method of claim 26, wherein the first primer is an exonuclease resistant primer.

28. The method of claim 27, wherein the first primer further comprises at least one phosphothioate modified intersubunit linkage at its 3′ terminus.

29. The method of claim 26, wherein the cyphers comprise a length ranging from about 5 nucleotides to about 10 nucleotides.

30. The method of claim 26, wherein the cyphers further comprise a nucleic acid molecule priming site.

31. The method of claim 26, wherein the cyphers further comprise at least one adapter sequence.

32. The method of claim 26, wherein the first primer further comprises a tag molecule.

33. The method of claim 32, wherein the tag molecule is biotin.

34. The method of claim 32 or 33, further comprising a purification step following the rolling circle amplification step, wherein the purification step isolates the strand of tandem nucleic acid molecules comprising multiple copies of the first target nucleic acid molecule or portion thereof via the tag molecule.

35. The method of claim 34, wherein after the purification step, the library of double-stranded circular bar-coded template molecules is re-used in a method for enriching a second target nucleic acid molecule.

36. The method of claim 26, wherein the plurality of double-stranded nucleic acid molecules is genomic DNA.

37. The method of claim 26, wherein the plurality of double-stranded nucleic acid molecules is human.

38. The method of claim 26, wherein the plurality of double-stranded nucleic acid molecules is obtained from a tumor sample, a blood sample, or a biopsy sample.

39. The method of claim 26, wherein the plurality of double-stranded nucleic acid molecules comprise a length ranging from about 100 to about 3,000 bases.

40. The method of claim 26, wherein target nucleic acid molecule comprises an oncogene, tumor suppressor gene, or fragment thereof.

41. The method of claim 40, wherein the tumor suppressor gene is TP53.

42. The method of claim 26, wherein the target nucleic acid molecule is enriched at least 10², 10³, 10⁴, 10⁵, 10⁶, 10⁷, 10⁸, or 10⁹-fold.

43. The method of claim 26, wherein step (a) further comprises a second primer specific for a first target nucleic acid molecule, wherein rolling circle amplification produces two strands of tandem nucleic acid molecules comprising multiple copies of the first target nucleic acid molecule or portion thereof.

44. The method of claim 43, wherein the second primer is antisense or sense to the first sense or antisense primer, respectively, wherein rolling circle amplification produces two complementary strands of tandem nucleic acid molecules comprising multiple copies of the first target nucleic acid molecule or portion thereof.

45. The method of claim 26, wherein step (a) further comprises three or more primers specific for a first target nucleic acid molecule.

46. The method of claim 26, wherein the method further comprises amplifying with a plurality of primers specific for a plurality of different target nucleic acid molecules.

47. The method of claim 26, further comprising:
(b) a second amplification step comprising amplification of the first target nucleic acid molecules or portions thereof and flanking 5′ and 3′ cyphers on each strand of tandem nucleic acid molecules produced from step (a); and
(c) sequencing the first target nucleic acid molecules or portions thereof produced from step (b).
